(19) United States
(12) Patent Application Publication
(10) Pub. No.: US 2018/0022668 A1
(43) Pub. Date: Jan. 25, 2018

(54) HYDROGENATION METHOD

(71) Applicant: UOP LLC, Des Plaines, IL (US)

(72) Inventors: Chad A. Williams, Des Plaines, IL (US); Patrick J. Bullen, Elmhurst, IL (US); Clint H. Vericker, Des Plaines, IL (US); Russell C. Schulz, Glen Ellyn, IL (US); Jose L. Miramontes, Madrid (ES)

(21) Appl. No.: 15/721,893

(22) Filed: Sep. 30, 2017

Related U.S. Application Data
(63) Continuation of application No. PCT/US2016/024412, filed on Mar. 28, 2016.
(60) Provisional application No. 62/140,862, filed on Mar. 31, 2015.

(51) Int. Cl. C07C 5/11; C07C 15/46; B01D 3/14; C07C 15/085; C10G 49/06

(52) U.S. Cl. CPC C07C 5/11; C07C 15/085; C10G 49/06; B01D 3/14; C07C 15/46

(57) ABSTRACT

The present subject matter relates generally to methods for treating an organic feed. More specifically, the present subject matter relates to methods for reducing the water content of an organic feed before the organic feed enters a hydrogenation zone, thereby by improving the activity, conversion, and life of the hydrogenation catalyst. The hydrogenation zone product stream is then sent to a phenol recovery zone.

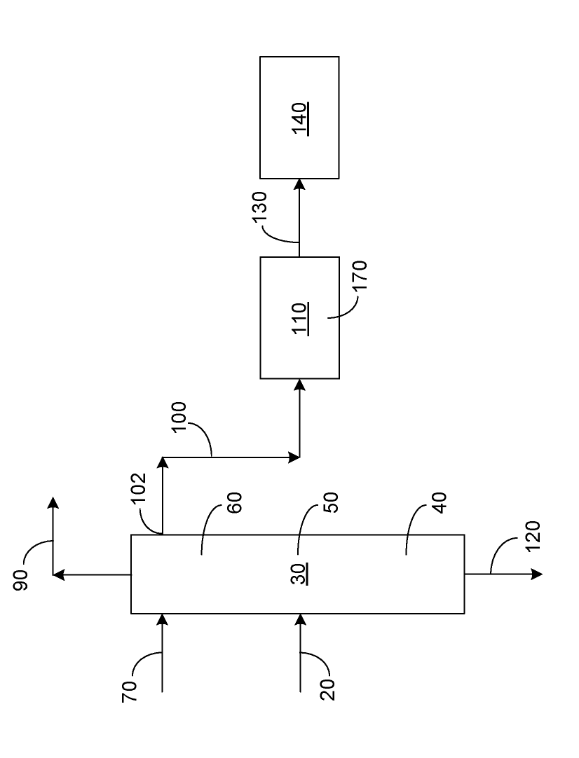

### CROSS-REFERENCE TO RELATED APPLICATIONS

This application is a Continuation of International Application No. PCT/US2016/024412 filed Mar. 28, 2016 which claims benefit of U.S. Provisional Application No. 62/140,862 filed Mar. 31, 2015, the contents of which cited applications are hereby incorporated by reference in their entirety.

### TECHNICAL FIELD

The present subject matter relates generally to methods for treating an organic feed. More specifically, the present subject matter relates to methods for reducing the water content of an organic feed before the organic feed enters a hydrogenation zone, thereby by improving the activity, conversion, and life of the hydrogenation catalyst.

### BACKGROUND

Hydrogenation catalysts in a phenol unit may be deactivated by undesired products such as water and sodium hydroxide carried over from an upstream extraction column. Sodium hydroxide and water can deactivate the hydrogenation catalyst which results in low catalyst activity, short catalyst cycle length, and the need to reactivate the catalyst.

Accordingly, it is desirable to develop methods for treating an organic feed by removing water before the organic feed enters the hydrogenation zone. Furthermore, other desirable features and characteristics of the present embodiment will become apparent from the subsequent detailed description and the appended claims, taken in conjunction with the accompanying drawings and this background.

### SUMMARY

A process for treating an organic feed includes introducing a feed stream containing at least one organic acid compound into a column, removing an organic product stream from the column having a reduced level of organic acid relative to the feed stream, and introducing the organic product stream to a hydrogenation zone. The organic feed stream may include cumene and alpha-methylstyrene and the organic acid compound may include phenol. The column is designed to remove water and salt from the organic feed stream. In one embodiment, the organic product stream contains 0.2 wt % to 0.5 wt % dissolved water. In another embodiment, organic product stream would contain 10 wt ppm to 200 wt ppm dissolved water. The organic product stream is a mixture comprising of 75-90 weight percent cumene and alphamethylstyrene. The organic product stream may also contain about 5 wt % to about 7 wt % phenol upon entering the hydrogenation zone. The organic product stream is sent to a hydrogenation zone containing at least one reactor having a hydrogenation catalyst. The hydrogenation zone thereby produces a hydrogenation zone product stream that may be sent to a phenol recovery zone. In another embodiment, the hydrogenation zone product stream that may be sent to an additional fractionation zone.

An advantage of the method for treating an organic feed is that the water content of the hydrocarbon stream is reduced before the hydrocarbon stream enters the hydrogenation zone.

Another advantage of the method for treating an organic feed is that the salt content of the hydrocarbon stream is reduced before the hydrocarbon stream enters the hydrogenation zone.

Reducing the water and salt content from the hydrocarbon feed before it enters the hydrogenation zone improves the activity, conversion, and life of the hydrogenation catalyst.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### Definitions

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C₁, C₂, C₃, Cn where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈, An where “n” represents the number of carbon atoms in the one or more aromatic molecules. Furthermore, a superscript “+” or “−” may be used with an abbreviated one or more hydrocarbons notation, e.g., C₃₊ or C₃₋, which is inclusive of the abbreviated one or more hydrocarbons. As an example, the abbreviation “C₃₊” means one or more hydrocarbon molecules of three or more carbon atoms.

As used herein, the term “zone” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

### BRIEF DESCRIPTION OF THE DRAWING

The drawing figures depict one or more implementations in accord with the present concepts, by way of example only, not by way of limitations. In the figures, like reference numerals refer to the same or similar elements.

The FIGURE is a schematic depiction of a method for treating an organic feed comprising a hydrogenation zone upstream of a phenol recovery unit.

### DETAILED DESCRIPTION

The following detailed description is merely exemplary in nature and is not intended to limit the application and uses of the embodiment described. Furthermore, there is no intention to be bound by any theory presented in the preceding background or the following detailed description.

The further description of the process of this invention is presented with reference to the attached drawing. The drawing is a simplified flow diagram of a preferred embodiment of this invention and is not intended as an undue limitation on the generally broad scope of the description provided herein and the appended claims. Certain hardware such as valves, pumps, compressors, heat exchangers, instrumentation and controls, have been omitted as not essential to a clear understanding of the invention. The use and application of this hardware is well within the skill of the art.

A process for treating an organic feed includes introducing a feed stream containing at least one organic acid compound into a column, removing an organic product stream from the column having a reduced level of organic acid relative to the feed stream, and introducing the organic product stream to a hydrogenation zone. The organic feed stream may include cumene and alpha-methylstyrene, and the organic acid compound may include phenol. The column is designed to remove water and salt from the organic feed stream. In one embodiment, the organic product stream contains 0.2 wt % to 0.5 wt % dissolved water. In another embodiment, organic product stream contains 10 wt. ppm to 200 wt. ppm dissolved water. The organic product stream may be a mixture comprising 75-90 wt % cumene and alphamethylstyrene. The organic product stream may also contain about 5 wt % to about 7 wt % phenol. The organic product stream may be sent to a hydrogenation zone containing at least one reactor having a hydrogenation catalyst. The hydrogenation zone thereby produces a hydrogenation zone product stream that may be sent to a phenol recovery zone. In another embodiment, the hydrogenation zone product stream that may be sent to an additional fractionation zone.

The overall process to which this invention pertains concerns the oxidation of a secondary alkylbenzene, for example, isopropylbenzene (cumene) isobutylbenzene, isoamylbenzene, 1-methyl-4-isopropylbenzene, p-diisopropylbenzene, p-diisobutylbenzene, 1-isopropyl-4-isobutylbenzene, cyclohexyl benzene, and the like, to form the corresponding hydroperoxide, i.e., isopropylbenzene hydroperoxide, isobutylbenzene hydroperoxide, isoamylbenzene hydroperoxide, 1-methyl-4-isopropylbenzene hydroperoxide, p-diisopropylbenzene hydroperoxide, p-diisobutylbenzene hydroperoxide, 1-isobutyl-4-isopropylbenzene dihydroperoxide, cyclohexylbenzene hydroperoxide, and the like. The present invention is particularly directed to a process for the preparation of a cumene feed for cumene oxidation from a fresh cumene stream and a recycle cumene stream containing trace quantities of at least one organic acid compound. The organic acid compound is selected from the group consisting of formic acid, acetic acid, benzoic acid, propionic acid, butyric acid and phenol.

The various embodiments described herein relate to methods for treating an organic feed using a column. In accordance with the present invention, the vertical, countercurrent contacting zone is preferably contained in a vessel such as a column 30, which has packing, trays or other means to provide countercurrent liquid-liquid extraction. The contacting zone is preferably operated at a pressure from about from about 0.1 kg/cm2(g) to about 5 kg/cm2(g) and a temperature from about 68° F. (20° C.) to about 122° F. (50° C.). However, other operating temperatures and pressures may be used in the practice of the present process, but preferably so long as the liquid phase is maintained.

Turning to the FIGURE, a feed stream 20 enters the column 30 which may be a cumene and alpha-methylstyrene column. The feed 20 in the example shown in the FIGURE includes cumene, alpha-methylstyrene, and phenol. However, it is contemplated that the feed may contain other hydrocarbon mixtures. For example, it is contemplated that the feed may contain acetone, organic acids, benzene, hydroxyacetone, 2-MBF, acetaldehyde, propionaldehyde, and heavy alkyphenols.

The column 30 comprises a lower portion 40, an intermediate portion 50, and an upper portion 60. The feed 20 enters the column 30 in the intermediate portion 50. A water stream 70 enters the column 30 in the upper portion 60 of the column 30. A portion of the first product stream 90 may be recycled back to the feed 20. The recycled product may be admixed with the feed 20 before entering the column 30, or the recycled product feed and the feed 20 may enter the column 30 at distinct inlets.

A second product stream 120 exits from the bottom of the column 30. The second product stream 120 comprises water, and sodium phenate.

A third product stream 100 comprising cumene, alpha-methylstyrene, and phenol may be taken from a side outlet 102 of the column 30. The third product stream 100 enters a hydrogenation zone 110. In one embodiment, the third organic product stream 100 contains about 0.2 wt % to about 0.5 wt % dissolved water. In another embodiment, the third organic product stream 100 contains about 10 wt ppm to about 200 wt ppm dissolved water. The third organic product stream 100 is a mixture comprising about 75 to about 90 weight percent cumene and alphamethylstyrene. The third organic product stream 100 may also contain about 5 wt % to about 7 wt % phenol.

In one example, the third organic product stream 100 may pass through a feed tank before entering the hydrogenation zone 110. In this example, the feed tank may reduce upsets in the feed.

The organic product stream 100 contains about 5 wt % to about 7 wt % phenol upon entering the hydrogenation zone 110. The hydrogenation zone 110 comprises at least one reactor. The pressure of the hydrogenation zone 110 is between about 2 kg/cm2(g) (28 psig) to about 25 kg/cm2(g) kPa (355 psig). The temperature of the hydrogenation zone 110 is between about 38° C. (100° F.) to about 150° C. (300° F.). The hydrogenation zone 110 contains at least one hydrogenation catalyst 170.

The hydrogenation catalyst 170 may be employed in a finely divided state or supported on a suitable base or carrier such as alumina, charcoal, silica alumina, silica gel, kieselguhr or similar materials. If the hydrogenation catalyst is supplied in the form of metal oxides, the catalyst can be reduced prior to use in the hydrogenation zone 110. The percentage of the metal in the catalyst can vary widely and may, for example, range from 0.1 to 17% or more. Any suitable hydrogenation catalyst 170 may be used in the hydrogenation zone 110. In one example, a hydrogenation catalyst using a hydrogenation metal such as palladium or nickel is suitable.

The hydrogenation zone product stream 130 is then sent to a phenol recovery zone 140. However, in another embodiment, the hydrogenation zone product stream may be sent to a fractionation zone before entering the phenol recovery zone 140.

It should be noted that various changes and modifications to the presently preferred embodiments described herein will be apparent to those skilled in the art. Such changes and modifications may be made without departing from the spirit and scope of the present invention and without diminishing its attendant advantages.

### SPECIFIC EMBODIMENTS

While the following is described in conjunction with specific embodiments, it will be understood that this description is intended to illustrate and not limit the scope of the preceding description and the appended claims.

A first embodiment of the invention is a process for treating an organic feed comprising introducing a feed stream containing at least one organic acid compound into a column; removing an organic product stream from the column having a reduced level of organic acid relative to the feed stream; and introducing the organic product stream to a hydrogenation zone. An embodiment of the invention is one, any or all of prior embodiments in this paragraph up through the first embodiment in this paragraph, wherein the organic feed comprises cumene and alpha-methylstyrene. An embodiment of the invention is one, any or all of prior embodiments in this paragraph up through the first embodiment in this paragraph, wherein the organic acid compound is phenol. An embodiment of the invention is one, any or all of prior embodiments in this paragraph up through the first embodiment in this paragraph, wherein the organic product stream contains 0.2 wt % to 0.5 wt % dissolved water. An embodiment of the invention is one, any or all of prior embodiments in this paragraph up through the first embodiment in this paragraph, wherein the organic product stream contains 10 wt ppm to 200 wt ppm dissolved water. An embodiment of the invention is one, any or all of prior embodiments in this paragraph up through the first embodiment in this paragraph, wherein the organic product is a mixture comprising of 75 wt % to 90 wt % cumene and alphamethylstyrene. An embodiment of the invention is one, any or all of prior embodiments in this paragraph up through the first embodiment in this paragraph, wherein the organic product stream contains about 5 wt % to about 7 wt % phenol. An embodiment of the invention is one, any or all of prior embodiments in this paragraph up through the first embodiment in this paragraph, wherein the column is operated at a pressure from about 0.1 kg/cm2(g) to about 5 kg/cm2(g). An embodiment of the invention is one, any or all of prior embodiments in this paragraph up through the first embodiment in this paragraph, wherein the column is operated at a temperature from about 20° C. to 50° C. An embodiment of the invention is one, any or all of prior embodiments in this paragraph up through the first embodiment in this paragraph, wherein the hydrogenation zone comprises at least one reactor. An embodiment of the invention is one, any or all of prior embodiments in this paragraph up through the first embodiment in this paragraph, wherein the pressure of the hydrogenation zone is between about 2 kg/cm2(g) (28 psig) to about 25 kg/cm2(g) kPa (355 psig). An embodiment of the invention is one, any or all of prior embodiments in this paragraph up through the first embodiment in this paragraph, wherein the temperature of the hydrogenation zone is between about 38° C. (100° F.) to about 150° C. (300° F.). An embodiment of the invention is one, any or all of prior embodiments in this paragraph up through the first embodiment in this paragraph, wherein a palladium hydrogenation catalyst is employed. An embodiment of the invention is one, any or all of prior embodiments in this paragraph up through the first embodiment in this paragraph, wherein a nickel hydrogenation catalyst is employed. An embodiment of the invention is one, any or all of prior embodiments in this paragraph up through the first embodiment in this paragraph, further comprising a hydrogenation zone product stream that may be sent to a phenol recovery zone. An embodiment of the invention is one, any or all of prior embodiments in this paragraph up through the first embodiment in this paragraph, further comprising a hydrogenation zone product stream that may be sent to an additional fractionation zone.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for treating an organic feed comprising:
introducing a feed stream containing at least one organic acid compound into a column;
removing an organic product stream from the column having a reduced level of organic acid relative to the feed stream; and
introducing the organic product stream to a hydrogenation zone.

2. The process of claim 1, wherein the organic feed comprises cumene and alpha-methylstyrene.

3. The process of claim 1, wherein said organic acid compound is phenol.

4. The process of claim 1, wherein the organic product stream contains 0.2 wt % to 0.5 wt % dissolved water.

5. The process of claim 1, wherein the organic product stream contains 10 wt ppm to 200 wt ppm dissolved water.

6. The process of claim 1, wherein the organic product is a mixture comprising about 75 wt % to about 90 wt % cumene and alphamethylstyrene.

7. The process of claim 1, wherein the organic product stream contains about 5 wt % to about 7 wt % phenol.

8. The process of claim 1, wherein the column is operated at a pressure from about 0.1 kg/cm2(g) to about 5 kg/cm2(g).

9. The process of claim 1, wherein the column is operated at a temperature from about 20° C. to 50° C.

10. The process of claim 1, wherein the hydrogenation zone comprises at least one reactor.

11. The process of claim 1, wherein the pressure of the hydrogenation zone is between about 2 kg/cm2(g) (28 psig) to about 25 kg/cm2(g) kPa (355 psig).

12. The process of claim 1, wherein the temperature of the hydrogenation zone is between about 38° C. (100° F.) to about 150° C. (300° F.).

13. The process of claim 1, wherein a palladium hydrogenation catalyst is employed in the hydrogenation zone.

14. The process of claim 1, wherein a nickel hydrogenation catalyst is employed in the hydrogenation zone.

15. The process of claim 1, further comprising sending a hydrogenation zone product stream from the hydrogenation zone to a phenol recovery zone.

16. The process of claim 1, further comprising sending a hydrogenation zone product stream from the hydrogenation zone to a fractionation zone.
